(19) United States
(12) Patent Application Publication
(10) Pub. No.: US 2023/0355780 A1
(43) Pub. Date: Nov. 9, 2023

(54) Synthetic Implantable Composition with Immunomodulatory Properties

(71) Applicant: Queen's University at Kingston, Kingston (CA)

(72) Inventors: Brian G. Amsden, Godfrey (CA); Fei Chen, Kingston (CA)

(21) Appl. No.: 18/142,878

(22) Filed: May 3, 2023

Related U.S. Application Data
(60) Provisional application No. 63/338,128, filed on May 4, 2022.

(51) Int. Cl. A61K 47/59; A61P 37/06; A61K 9/00

(52) U.S. Cl. CPC A61K 47/593; A61P 37/06; A61K 9/0024

(57) ABSTRACT

Implantable compositions are based on polymers bearing pendant groups on the polymer backbone functionalized with short chain fatty acid groups, such as one or more of acetate, n-propionate, and n-butyrate by a degradable linkage. Upon a composition being implanted in a subject, such as a human, degradation of degradable linkage liberates the short chain fatty acid groups. The liberated short chain fatty acid groups may then participate in biochemical processes in the subject, which may produce a therapeutic benefit such as resolving inflammation, promoting tissue healing, and/or promoting tolerating self-antigens and neoantigens.

### RELATED APPLICATION

This application claims the benefit of the filing date of Application No. 63/338,128, filed on May 4, 2022, the contents of which are incorporated herein by reference in their entirety.

### FIELD

The invention relates generally to the field of implantable compositions. More particularly, the invention relates to implantable compositions based on polymers having a polymer backbone functionalized with groups with immunomodulatory properties. The compositions may be used for delivery and sustained release of groups with immunomodulatory properties as well as drugs and therapeutic agents to an anatomical site of a subject.

### BACKGROUND

A primary concern in developing biomedical devices based on polymer biomaterials is the nature and severity of the host response following implantation. Implantation into tissue initiates inflammation followed by a wound-healing response. The sequence of events is a continuum and can be considered to involve an acute inflammatory phase, followed by a chronic inflammatory phase, and if the material persists for a prolonged period in the tissue, a granulation phase that ultimately results in the formation of a scar-like fibrotic tissue surrounding the implanted device. This process isolates the biomaterial from the rest of the body, and can render the device nonfunctional.

The acute phase is characterized by the adsorption of blood proteins and proteins and other molecules released by damaged cells to the material surface and by the presence of neutrophils and lasts for 1-3 days. The adsorbed proteins form a provisional tissue matrix on the biomaterial surface. The chronic phase is characterized by the presence of macrophages, which play pivotal roles in the duration and outcome of the granulation phase. The macrophages are either tissue resident or arise from monocytes that extravasate from blood vessels, migrate to the material surface, and adhere to the provisional matrix. Once at the site, the macrophages become activated. The activated macrophages are a heterogeneous population of cells that can be broadly classified into either a pro-inflammatory (M1) phenotype, or a pro-regenerative (M2) phenotype. A higher percentage of M2s at the site has been associated with improved response outcomes, such as a reduction in the fibrous capsule layer thickness and improved tissue healing rates (Klopfleisch et al., 2017). It is therefore of interest to create polymer biomaterials that can modulate macrophage polarization towards an M2 phenotype.

### SUMMARY

One aspect of the invention relates to a composition, comprising: polymer having a polymer backbone bearing pendant groups wherein at least a portion of the pendant groups comprise one or more short chain fatty acid groups; wherein the one or more short chain fatty acid groups are conjugated to the pendant groups of the polymer backbone by degradable linkages.

The degradable linkages may be degradable by one or more of hydrolysis, oxidation, and enzymatic action.

The degradable linkages may comprise one or more of ester, anhydride, carbonate, amide, thioester, urethane, acetal, disulfide, and orthoester.

In some embodiments, the degradable linkages comprise ester linkages.

In some embodiments, the portion of the pendant groups that are conjugated with the one or more short chain fatty acid groups is from 1% to 100%.

In some embodiments, the one or more short chain fatty acid groups comprises one or more of acetate, propionate, and butyrate.

In some embodiments, the polymer is selected from a vinyl polymer, a polyester, and an aliphatic polycarbonate.

In some embodiments, the polymer is an aliphatic polycarbonate.

In some embodiments, the polymer is selected from poly(vinyl alcohol), poly(5-hydroxy caprolactone), poly(5-hydroxy caprolactone-co-lactide) and combinations with glycolide and/or trimethylene carbonate, poly(5,6-hydroxy tetramethylene carbonate), poly(5,5-dihydroxy trimethylene carbonate), and poly(1,2-glycerol carbonate).

In some embodiments, the polymer is degradable.

In some embodiments, a degradation rate of the polymer is controlled according to a molar ratio of repeating unit in the polymer.

In some embodiments, the polymer is poly(2-hydroxy trimethylene carbonate-trimethylene carbonate) (poly(HT-T)).

In some embodiments, the short chain fatty acid comprises butyrate.

In some embodiments, the composition comprises: 2-butyrate trimethylene carbonate (BtT); 2-hydroxy trimethylene carbonate (HT); and trimethylene carbonate (T).

In some embodiments, the BtT, HT, and T are present in a ratio within the range of about 25-15-60 to about 10-30-60.

In some embodiments, the composition comprises or is part of a biomedical device that is implantable and degradable in a subject.

In some embodiments, degradation of the degradable linkage liberates a group comprising at least the one or more short chain fatty acid from the polymer backbone.

In some embodiments, degradation of the degradable linkage comprises an intramolecular cyclization reaction that cleaves the polymer backbone forming soluble oligomers bearing the one or more short chain fatty acid group.

In some embodiments, degradation of the degradable linkage liberates a group comprising at least the one or more short chain fatty acid from the polymer backbone; wherein the group comprising at least the one or more short chain fatty acid liberated from the polymer backbone induces an immunomodulatory response in the subject.

In some embodiments, the immunomodulatory response in the subject comprises macrophage polarization towards an M2 phenotype in tissue.

In some embodiments, the biomedical device comprises a drug delivery vehicle and the polymer further comprises at least one drug.

In some embodiments, the at least one drug comprises at least one of a therapeutic compound, a pharmaceutical agent, a biopharmaceutical agent, a bioactive agent, a medicament, an antineoplastic agent, a hormone, a peptide, a protein, a nucleic acid, a vector, a virus, an antigen, and an antibody.

Another aspect of the invention relates to an implantable biomedical device comprising a composition as described herein.

Another aspect of the invention relates to a method for delivering one or more short chain fatty acid group to a subject, comprising: implanting in an anatomical site, organ, or tissue of the subject a composition as described herein; wherein degradation of the degradable linkage liberates a group comprising at least the one or more short chain fatty acid from the polymer backbone and delivery of at least the one or more short chain fatty acid group to the anatomical site, organ, or tissue of the subject.

Another aspect of the invention relates to a method for resolving inflammation, promoting tissue healing, and/or promoting tolerating self-antigens and neoantigens in a subject, comprising: implanting in an anatomical site, organ, or tissue of the subject a composition as described herein; wherein degradation of the degradable linkage liberates a group comprising at least the one or more short chain fatty acid from the polymer backbone and delivery of at least the one or more short chain fatty acid group to the anatomical site, organ, or tissue of the subject; wherein the liberated group comprising at least the one or more short chain fatty acid resolves inflammation, promotes tissue healing, and/or promotes tolerating self-antigens and neoantigens in the anatomical site, organ, or tissue of the subject.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1A is a diagram showing a synthesis route to P(BtT-HT-T), according to one embodiment.

FIG. 1B is a diagram showing a representative degradation route of P(BtT-HT-T), according to one embodiment.

FIG. 2 is a diagram showing a structure and 1H NMR spectrum of P(BtT-HT-T)-10-30-60 along with peak assignments, according to one embodiment.

FIGS. 3A-3C are plots of change in (A) mass, (B) pH of the degradation medium, and (C) number average molecular weight (Mₙ) with time of three polymers during in vitro degradation.

FIGS. 4A-4C are plots of change in monomer composition of polymers with time during in vitro degradation: (A) P(HT-T)-40-60, (B) P(BtT-HT-T)-10-30-60, and (C) P(BtT-T).

FIG. 5 shows photographs of representative polymer implants in the subcutaneous tissue of rats at weeks 2 and 6 after implant.

FIGS. 6A-6C are plots of change in monomer composition of polymers with time during in vivo degradation: (A) P(HT-T)-30-70, (B) P(BtT-HT-T)-15-25-60, and (C) P(BtT-T)-40-60.

FIG. 7 is a plot of ratio of M1 macrophages (CD68+CCR7+) present to all macrophages (CD68+) present in immunostained tissue of rats that received polymer implants, wherein * represents statistical significance at the 95% confidence level, ** represents statistical significance at the 99% confidence level.

FIG. 8 is a plot of ratio of M2 macrophages (CD68+CD163+) present to all macrophages (CD68+) present in immunostained tissue of rats that received polymer implants, wherein * represents statistical difference at the 95% confidence level, ** represents a statistical difference at the 99% confidence level, and *** represents statistical difference at the 99.9% confidence level.

FIG. 9 is a plot of thickness of the fibrous capsule layer surrounding polymer implants at 2 and 6 weeks post-implantation in rats.

FIG. 10 is a plot of mass fraction of initially loaded amount of octreotide acetate released in vitro from polymers of varying composition, wherein percentages in brackets are the initial w/w % loading of octreotide acetate in the polymers.

### DETAILED DESCRIPTION OF EMBODIMENTS

Described herein are implantable compositions comprising polymers bearing pendant short chain fatty acids. The short chain fatty acids may be conjugated to the polymer backbone by degradable linkages. The linkages may be degradable by one or more of hydrolysis, oxidation, and enzymatic action. Any of a number of degradable linkages known to those skilled in the art may be employed including but not limited to ester, anhydride, carbonate, amide, thioester, urethane, acetal, disulfide or orthoester. The conjugated short chain fatty acids may be of one type, or they may be a mix of more than one type. For example, short chain fatty acids, such as, e.g., one or more of acetate, propionate (e.g., n-propionate), and butyrate (e.g., n-butyrate), may be conjugated to the pendant hydroxyl groups of the polymer backbone. According to embodiments the degree of functionalization of polymers may be varied. Some embodiments may be fully (e.g., 100%) or almost fully (e.g., 90% to 99%) functionalized such that substantially all pendant hydroxyl groups are conjugated to a short chain fatty acid, whereas other embodiments may be less functionalized such that fewer (e.g., less than 100% to 1%) of the pendant hydroxyl groups are conjugated to a short chain fatty acid. For example, in various embodiment at least 25%, or at least 50%, or at least 75%, or at least 90% of the pendant hydroxyl groups may be conjugated to a short chain fatty acid. In some embodiments 10% to 25% of the pendant hydroxyl groups may be conjugated to a short chain fatty acid.

In various embodiments, the composition may be at least partially degradable in a biological setting (e.g., in an organism, i.e., in vivo) wherein degradation liberates the short chain fatty acids in a form where they are bioactive and yields products that are not harmful (i.e., they are non-irritating and/or non-toxic and do not cause a substantive change in pH) to the biological setting. A composition that is at least partially degradable in a biological setting may also be referred to herein as biodegradable.

According to embodiments, polymers with pendant hydroxyl groups on the polymer backbone that may be used include, but are not limited to, those based on polyesters, vinyl polymers, or aliphatic polycarbonates. Examples include, but are not limited to, poly(vinyl alchohol), poly(5-hydroxy caprolactone), poly(5-hydroxy caprolactone-co-lactide) and other copolymer combinations, such as, for example, combinations with glycolide and trimethylene carbonate, poly(5,6-hydroxy tetramethylene carbonate), poly(5,5-dihydroxy trimethylene carbonate), and poly(1,2-glycerol carbonate). Whereas the term “polymer” is used generally herein, it will be appreciated that in some embodiments a polymer may be a “copolymer”, i.e., a polymer derived from more than one species of monomer. In some embodiments, the polymers may be degradable. A non-limiting example of a degradable polymer is poly(2-hydroxy trimethylene carbonate-trimethylene carbonate) (poly(HT-T)).

In some embodiments, implantable compositions as described herein may be implanted in an anatomical site (e.g., a body cavity, tissue, organ, etc.) of a subject, such as a human. Degradation of an implanted composition may include hydrolytic liberation of the conjugated short chain fatty acids. In some embodiments the liberated short chain fatty acid may remain conjugated to a polymer repeating unit (e.g., it may remain linked to a carbonate monomer in the case of an aliphatic polycarbonate). The liberated short chain fatty acids may then participate in biochemical processes in the subject, which may produce a therapeutic benefit. In some embodiments, upon a composition as described herein being implanted in a subject, degradation may also include degradation of the polymer. In some embodiments, degradation of the polymer may be based on water solubility of the oligomeric products formed from an intramolecular cyclization reaction of the pendant hydroxyl groups of the polymer repeating units.

Short chain fatty acids such as acetate, n-propionate, and n-butyrate may enhance M2 macrophage polarization in tissue surrounding the implanted degradable polymer, which provides benefits such as resolving inflammation, promoting tissue healing, and tolerating self-antigens and certain neoantigens. These short chain fatty acids may be produced by microbes in the large intestine and have been demonstrated to modulate inflammation associated with inflammatory bowel disease. For example, these short chain fatty acids have been shown to reduce the production of inflammatory markers by neutrophils and monocytes (Cox et al., 2009; Vinolo et al., 2009, 2010; Tedelind et al., 2007), while butyrate has been reported to promote the production of anti-inflammatory markers (Liu et al., 2012). Moreover, butyrate has been reported to modulate intestinal macrophage polarization (Ji et al., 2016).

One aspect of the invention relates to implantable medical devices based on compositions as described herein, wherein pendant hydroxyl groups on the polymer backbone are functionalized with short chain fatty acids. Embodiments may have utility in applications such as controlled sustained release of fatty acids at an anatomical site, with release rate controllable through adjustment of the polymer repeating unit composition. Further, in certain embodiments wherein the compositions are based on degradable polymers, the compositions may also be used to deliver therapeutic agents (e.g., drugs, peptides, etc.) to an anatomical site. The degradable polymer may be loaded with one or more therapeutic agent to provide controlled sustained release of the therapeutic agent at an anatomical site as degradation of the polymer proceeds. Methods and protocols for loading polymers with therapeutic agents are known in the art, and described in the below Examples.

According to embodiments, when a composition is implanted in a subject, hydrolytic liberation of short chain fatty acid groups (e.g., one or more of acetate, n-propionate, and n-butyrate) conjugated to pendant hydroxyl groups of the polymer backbone results in elevated levels of the short chain fatty acids and enhanced M2 polarization in tissue surrounding the implanted degradable polymers. Compositions and methods described herein provide a mechanism by which amounts of such short chain fatty acids in tissues and organs may be significantly increased over amounts that may be naturally produced, thereby providing therapeutic benefits. Moreover, compositions and methods described herein enable such short chain fatty acids to be delivered to anatomical sites and tissues where the short chain fatty acids would not otherwise be naturally produced.

According to embodiments, conjugation of a short chain fatty acid to a polymer backbone provides a mechanism for adjusting the degradation rate of the polymer as a result of a reduced water solubility of the oligomeric products formed from the intramolecular cyclization reaction of the pendant hydroxyl groups of the polymer repeating units. For example, selecting the degree of functionalization (e.g., from 1% to 100%) with short chain fatty acid groups may provide a mechanism for controlling the degradation rate of the polymer and the amount of one or more short chain fatty acids delivered to a subject. The degradation does not lead to significant lowering of the local pH, which remains at or near physiologic pH in vitro.

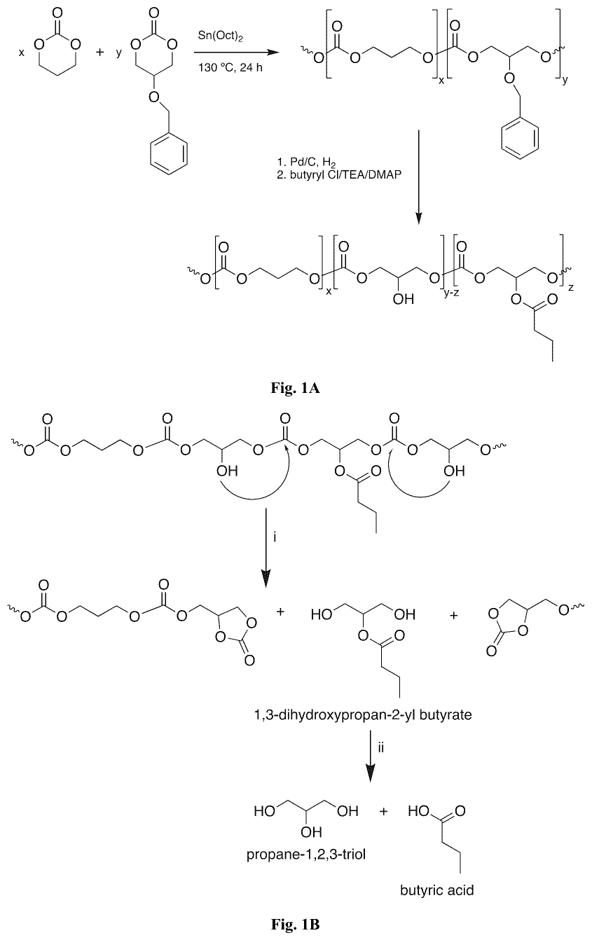

In one embodiment, n-butyrate was conjugated to the pendant hydroxyl groups of the backbone of an aliphatic polycarbonate, poly(2-hydroxy trimethylene carbonate-trimethylene carbonate) (poly(HT-T)), to produce P(BtT-HT-T). A synthesis route is shown in FIG. 1A. As described in the Example below, the composition was implanted subcutaneously in subjects (rats), and butyrate liberated from the polymer backbone as a result of the intramolecular cyclization degradation reaction induced a polarization of macrophages towards an M2 phenotype around the implanted polymer, resulting in a greater population of pro-regenerative M2 macrophages in the inflammatory zone. A direct benefit is that the increase in M2 cell population results in a reduced thickness of the fibrous collagen capsule formed around the implanted polymer. Without wishing to be bound by theory, a representative degradation route of P(BtT-HT-T) is shown in FIG. 1B. A first degradation step (i) proceeds via intramolecular cyclization leading to the release of bioactive 1,3-dihydroxypropan-2-yl butyrate, followed by (ii) the subsequent hydrolysis of 1,3-dihydroxypropan-2-yl butyrate to generate propane-1,2,3-triol (glycerol) and bioactive butyric acid.

Another aspect of the invention relates to a method for delivering a short chain fatty acid such as one or more of acetate, n-propionate, and n-butyrate to an anatomical site, organ, or tissue of a subject, comprising implanting in the anatomical site, organ, or tissue of the subject a composition comprising a polymer bearing pendant hydroxyl groups on the polymer backbone that are conjugated with the one or more short chain fatty acids by a degradable linkage, wherein degradation includes hydrolytic liberation and delivery of the conjugated short chain fatty acids to the anatomical site, organ, or tissue of the subject.

Another aspect of the invention relates to a method for resolving inflammation, promoting tissue healing, and/or promoting tolerating self-antigens and neoantigens in an anatomical site, organ, or tissue of a subject, comprising implanting in the anatomical site, organ, or tissue of the subject a composition comprising a polymer bearing pendant hydroxyl groups on the polymer backbone that are conjugated with the one or more short chain fatty acids acids by a degradable linkage, wherein degradation includes hydrolytic liberation and delivery of the conjugated short chain fatty acids to the anatomical site, organ, or tissue of the subject, wherein the liberated short chain fatty acids resolve inflammation, promote tissue healing, and/or promote tolerating self-antigens and neoantigens in the anatomical site, organ, or tissue of the subject.

Embodiments will be further described by way of the following non-limiting Examples.

### EXAMPLES

### 1. Materials

5-benzyloxy-trimethylene carbonate (BT) was purchased from Obiter Research, LLC (Champaign, USA), trimethylene carbonate (T) was obtained from Leapchem in Hangzhou, China. Stannous 2-ethylhexanoate (Sn(Oct)₂) (96% purity) and butyryl chloride was obtained from Alfa Aesar, Canada. Palladium on activated carbon (10 wt % (Pd/C)), palladium hydroxide over carbon (20 wt % (Pd(OH)₂/C)) and deuterated methyl sulfoxide (DMSO-d6) were purchased from Acros Organics, New Jersey, USA. Hydrogen gas (H₂) (99.99% purity) was from Linde Canada Ltd, Ontario. 100 mL stainless steel pressure vessel was purchased from Parr Instrument Company, USA. Dichloromethane (DCM), toluene, tetrahydrofuran (THF) and triethylamine (TEA) was purchased from Fisher Scientific, Canada. Anhydrous THF was prepared by mixing with activated 4 Å molecular sieves (10 w/v %) for 48 h, all other reagents were used as received. Phosphate buffered saline (pH 7.4) (PBS 1x) was prepared by dissolving phosphate-buffered saline powder concentrate (Fisher BioReagents®) in deionized water at 9.9 mg/mL and the pH was adjusted with 1 M HCl to 7.4.

### 2. Methods

### Synthesis of poly(5-benzyloxy trimethylene carbonate-co-trimethylene carbonate) (P(BT-T))

P(BT-T) with a varying mol % BT were prepared through ring-opening polymerization. The following is a representative polymerization to obtain a copolymer with 40 mol % BT composition (P(BT-T)-40-60). In detail, BT (8 g, 0.0384 mol) and T (5.884 g, 0.0576 mol) were loaded into a 20 mL flame-dried ampoule and the monomers freeze-dried (−46° C., 0.2 mbar) overnight before 8.5 mg of Sn(Oct)₂ catalyst was added. The ampoule was purged with argon and then evacuated at a reduced pressure of 28 kPa for 30 s and finally flame-sealed. The ampoule was placed in a 130° C. oven for 24 h and polymerization halted by cooling down to 4° C. in a refrigerator. The reaction mixture was then first dissolved in 15 mL of THF and precipitated in 100 mL of methanol.

### Synthesis of poly(2-hydroxy trimethylene carbonate-co-T) (P(HT-T))

P(HT-T) was prepared through debenzylation of P(BT-T). The following is a representative procedure to obtain P(HT-T) with a 40% HT composition. 10 g of P(BT-T)-40-60 was first dissolved in 16 mL of THF. 900 mg of 10% Pd/C and 900 mg of 20% Pd(OH)₂/C were loaded into a Parr pressure vessel under N₂. The pre-dissolved polymer solution was added into the vessel under N₂, another 40 ml THF with 14 mL methanol were introduced into the vessel to reach a final THF/methanol ratio of 4:1(v/v). The vessel was purged with H₂ to 120 psi three times to remove 02 from the reactor and then the vessel was sealed and kept under 120 psi H₂ for 28 h at room temperature with a stirring rate of 500 rpm. Finally, the reaction mixture was filtered through Celite 500 to remove the catalysts, and the solvent was evaporated on a rotary evaporator to yield the polymer. The polymer was re-dissolved in THF and precipitated in diethyl ether and dried under vacuum at room temperature for two days before being freeze-dried at −46° C. and 0.3 mbar for another two days to obtain the final polymer.

### Synthesis of poly(2-butyrate trimethylene carbonate-co-T) (P(BtT-T))

A butyrate pendant group was conjugated to the P(HT-T) backbone by reaction of butyryl chloride with the hydroxyl group of P(HT-T). Different degrees of substitution were achieved by adjusting the amount of butyryl chloride. A representative reaction condition to achieve 100% substitution is described here. P(HT-T) with a HT composition of 40 mol % (P(HT-T)-40-60) (2 g, 7.373 mmol of —OH) was dissolved in 13 mL of anhydrous THF in a 20 mL glass vial by vortexing then transferred into a flame-dried 100 mL round bottom flask cooled with ice, triethylamine (TEA) (2.1 equiv., 15.49 mmol, 2.16 mL) was added by syringe first, followed by butyryl chloride (2 equiv., 14.75 mmol, 1.53 mL) dropwise by syringe under N₂ atmosphere. The reaction mixture was stirred for 6 h then concentrated on a rotary evaporator, re-dissolved in 45 mL of ethyl acetate, cooled to −20° C. in a freezer for 2 h and then centrifuged at 3000 rpm for 3 min. The supernatant was collected by decanting, then concentrated to 5 mL on a rotary evaporator, and then finally precipitated into 40 mL of ethanol. The polymer was further washed with ethanol (40 mL×3 times). The final polymer was dried under vacuum for two days then freeze-dried at −46° C. at 0.3 mbar for two days.

### Polymer Characterization

The composition of the copolymer was calculated from 1H-NMR spectra collected in DMSO-d6 on a Bruker Avance 400 MHz NMR with peak shifts referenced using an internal tetramethylsilane standard. The composition was calculated by comparing the integration of the characteristic peaks from T (—CH₂ at 1.95 ppm), HT (—CH—OH at 3.92 ppm), and BtT (—CH₃ at 0.87 ppm). The number average molecular weight (Mₙ) and dispersity (D) were assessed via gel permeation chromatography (GPC) (Waters 2695). The system was equipped with a differential refractive index detector (RI) and an automatic sample injection and delivery module (Waters). The samples were prepared at a concentration of 4 mg/mL in HPLC grade THF and were filtered with a 0.22 μm syringe filter (Chromatographic Specialties Inc. Canada) before being injected into the column. The eluent, THF, was set at a flow rate of 0.3 mL s⁻¹ at 40° C. The separation was done using four columns (Waters) [4×Styragel HR 4 THF (300×4.6 mm)]. Linear polystyrene standards (Mₙ=890 to 3.28×10⁶ g/mol) were used to determine the Mₙ and D using Empower 2 software (Waters). A Mettler Toledo DSC1 system was used to measure the glass transition temperature (Tg) of the polymers. The samples were run at a heating and cooling rate of 10° C./min using the following temperature program. The samples were first cooled from 25 to −80° C. and held for 1 min at that temperature. This cycle was followed by heating from −80 to 100° C. with no hold time and then cooling from 100 to −80° C. Finally, the sample was heated from −80 to 100° C. The glass transition temperature was obtained from the inflection point of the second heating cycle.

### In Vitro Hydrolytic Degradation

Samples of approximately 50-60 mg of P(HT-T)-40-60, P(BtT-T)-40-60 or P(BtT-HT-T)-10-30-60 in the shape of a sphere with an average diameter of 4.5±0.3 mm were placed in 4 mL glass vials of known weight with 4 mL of PBS (pH 7.4) (n=3 for each composition at each time point). The vials were placed in a Thermomixer and agitated at 100 rpm at 37° C. with the PBS refreshed twice a week. Three vials for each composition were analyzed at different time points over 12 weeks. The supernatant was collected and its pH measured. The remaining solid polymer in each vial was rinsed with distilled water twice then lyophilized. The mass loss was calculated by the following equation:

$$Mass loss = \frac{initial mass - final mass}{initial mass} \times 100 \%$$

### In Vivo Studies

The polymers (60-80 mg, spherical with a diameter of 5.3±0.2 mm) for in vivo implantation studies were first washed with 70% ethanol twice then UV sterilized inside a biological safety cabinet for 30 min and finally rinsed with sterile PBS three times (˜2 min each time). The animal experiments were performed in accordance with the guidance of the Canadian Council on Animal Care code of ethics governing animal experimentation (protocol Amsden 2015-1627). A total of 6 male Wistar rats (Charles River), weighing approximately 300-400 g were used. The rats were anesthetized with 2% isoflurane (Baxter Corp.) in oxygen at a flow rate of 0.2 mL/min of 02. At a level of surgical anesthesia, the dorsal area of the rats was shaved, the shaved area was disinfected with Hibitane, and a 1.5 cm long longitudinal incision was made and a pocket formed between the skin and the underlying tissue. The pre-sterilized polymer (2 samples per each composition per rat) was inserted into the pocket with the aid of a sterile spatula and the pocket closed with sutures. The implanted polymers were excised on weeks 1, 2, 6. Each animal was anesthetized using isoflurane (5% in oxygen) and then humanely euthanized with a lethal dose of Euthanyl® (120 mg/kg body weight). Fur on the backs of the rats surrounding the implant sites was removed with electric trimmers and straight razors. An incision was made through the skin along the length of the spine, and perpendicularly at the top (neck) and bottom (base of spine). Skin covering the entire back was carefully peeled to either side from the underlying muscle layer. The implants were visualized on the underside of the skin, held in place by a thin, translucent layer of fascia. Using surgical scissors, the sections of skin containing the implants were explanted with a small amount (approximately 2 mm) of the surrounding tissue.

### Histological and Immunohistochemical Analyses

Cryomolds were prepared by adding a thin (1 mm) layer of OCT compound to a mold cavity. Explanted tissue was then put into the cryomold then covered with more OCT compound. The samples were snap frozen in liquid nitrogen and then cryo-sectioned to 5 μm perpendicular to the skin and fixed in 4% paraformaldehyde.

### Masson's Trichrome Staining

Frozen sections were rinsed with distilled water and then stained with Masson's trichrome (Polysciences, Inc.) according to the manufacturer's instructions. Stained sections were imaged on an EVOS cell imaging system (10× magnification), and the collagen capsule thickness at the basal margin was quantified from the average of five to eight measurements using Zeiss Zen v. 2.3 software.

### Immunohistochemistry Staining

The presence and polarization of macrophages surrounding the subcutaneously implanted polymers was characterized by immunohistochemistry (IHC). Frozen sections were rinsed with distilled water, and blocked with 5% goat serum in tris buffered saline pH 7.4 (TBS) with 0.2% Tween-20 (TBS-T) for 30 minutes at room temperature. The serum solution was replaced with primary antibodies diluted in TBS-T with 1% bovine serum albumin (BSA) per Table 1 and incubated overnight at 4° C. Anti-CD68 was used to detect all macrophages and was used in combination with anti-CCR7 to detect M1-polarized macrophages and anti-CD163 to detect M2c-polarized macrophages. Following primary antibody incubation, the sections were rinsed three times with TBS, and incubated with secondary antibodies diluted in TBS-T with 1% BSA according to Table 1 for 1 h at room temperature. The secondary antibody solutions were removed, and the sections were rinsed three times in TBS. The sections were mounted with fluoroshield mounting medium with DAPI (Abcam). All imaging was performed with a Zeiss Axio Imager M1 microscope.

TABLE 1
Summary of primary and secondary antibodies used for IHC

| | | | | Conc. |
|---|---|---|---|---|
| Antibody | Clone | Supplier (Cat. #) | Dilution | (μg/mL) |
| Mouse anti-CD68 | ED1 | Bio-Rad | 1:200 | 5.0 |
| | | (MCA341R) | | |
| Rabbit anti-CCR7 | Y59 | Abcam | 1:100 | 10.0 |
| | | (ab32527) | | |
| Mouse anti-CD163 | ED2 | Bio-Rad | 1:100 | 6.7 |
| | | (MCA342GA) | | |
| Goat anti-mouse | Polyclonal | Abcam | 1:500 | 4.0 |
| IgG Alexa Fluor 488 | | (ab150113) | | |
| Goat anti-rabbit | Polyclonal | Sigma | 1:250 | 8.0 |
| IgG CF555 | | (SAB4600068) | | |

Macrophage density and polarization was quantified at the basal edge of the implanted polymers. At least three non-overlapping images (40× magnification) from the basal margin were taken for each sample, and the total number of macrophages (CD68+ cells), M1-polarized macrophages (CD68+CCR7+), and M2c-polarized macrophages (CD68+CD163+) were counted (ImageJ v. 1.46r and Zeiss Zen v. 2.3 software).

### In Vitro Peptide Release

P(BtT-HT-T) were prepared with a target Mₙ of 4000 Da and two different compositions: P(BtT-HT-T)-25-15-60 and P(BtT-HT-T)-10-30-60. To load the peptide, a weighed amount of polymer was added to a 1.5 mL Eppendorf tube. Octreotide acetate dissolved in methanol (10 mg/mL) was then added to the tube and placed under vacuum to remove the methanol. Once dry, the octreotide and polymer were manually stirred to disperse the octreotide acetate particles in the polymer. The polymer and octreotide acetate mixture was then transferred to new pre-weighed Eppendorf tubes. The tubes containing the polymer plus octreotide acetate mixture were then weighed to determine the initial mass of mixture present. 1.4 mL of phosphate-buffered saline (PBS) with a pH of 7.4 was added to 1.5 mL Eppendorf tubes containing approximately 30 mg of octreotide acetate-loaded polymer and the tubes then placed on an orbital mixer in a 37° C. oven. At predetermined time points, 1.2 mL of PBS was removed for analysis, and replenished with fresh PBS. The concentration of octreotide acetate in the solution was measured with an Agilent 1260 reversed phase high performance liquid chromatography system (HPLC) using a Nova Pak 2.9×150 mm C18 column. The injection volume was 20 μL. A gradient elution going from 25% acetonitrile in water with 0.1% v/v TFA to 35% acetonitrile in water with 0.1% v/v TFA over 20 minutes was used with a wavelength detection set to 215 nm. The concentration was obtained from a seven-point calibration curve ranging from 0.005 to 1 mg/mL prepared from serial dilutions of peptide in PBS done in duplicate. Once the release was complete, the remaining polymer and octreotide were dissolved in acetone. MilliQ water was added to extract the octreotide and precipitate the polymer. The tube was then centrifuged, and the liquid phase collected. This process was repeated three times. The liquid phase collected was lyophilized, and the dry material remaining was resuspended in 2 mL of pH 7.4 PBS for HPLC analysis to determine the amount of unreleased octreotide.

### Statistical Analysis

All data are presented as mean±standard deviation. Comparisons were made by two-way ANOVA. Differences between polymer type at each time point were determined by Tukey's multiple comparisons test. Differences were considered significant for p-values<0.05. All statistical analysis was performed with GraphPad Prism 9.

### 3. Results

### Copolymer Synthesis and Characterization

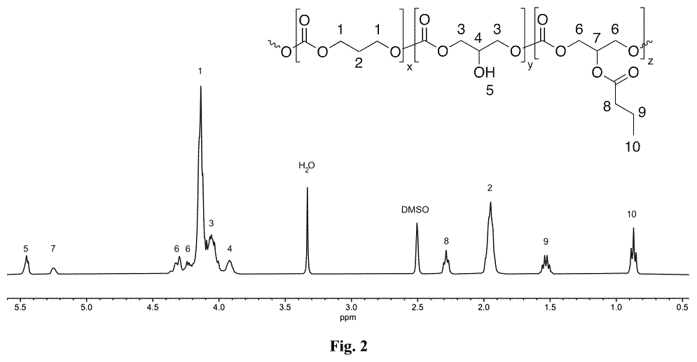

With the aim of preparing an amorphous aliphatic polycarbonate with a pendant butyrate group, P(BT-T) was first synthesized by bulk ring-opening polymerization in the presence of Sn(Oct)₂ catalyst, followed by removal of benzyl-protecting groups using Pd/H₂ catalyzed reaction to form P(HT-T). Finally, a butyrate pendant group was attached by the reaction of butyryl chloride with some or all of the hydroxyl pendant groups of the repeating HT units in the copolymer (FIG. 1). The degree of butyrate functionalization was adjusted by controlling the amount of butyryl chloride used in the conjugation reaction. A summary of the physical and chemical properties of the polymers prepared for the in vitro degradation study is given in Table 2, while the chemical structure and corresponding ¹HNMR spectrum for P(HT-BtT-T) with peak assignments is presented in FIG. 2.

Moderately high molecular weight polymers were obtained, with number average molecular weights (Mₙ) between 15 to 18 kDa. Further, with dispersities of approximately 1.3, the polymers had relatively narrow molecular weight distributions. All the polymers had low glass transition temperatures and were amorphous. Complete conversion of the pendant OH groups on the P(HT-T) to butyrate groups resulted in an increase in glass transition temperature, Tg, from −11 to −4° C., due to the increased molecular size of the pendant group. Partial conversion of the OH group resulted in an intermediate value for the Tg.

TABLE 2
Properties of polymers examined for in vitro degradation.

| | Target | Product | | | |
|---|---|---|---|---|---|
| | monomer | monomer | Mₙ | | T_{g} |
| Polymer | molar ratio | molar ratio | (Da) | Ð | (° C.) |
| P(HT-T)-40-60 | 40-60 | 40-60 | 17708 | 1.28 | −11 |
| P(BtT-T)-40-60 | 40-60 | 39-61 | 18155 | 1.33 | −4 |
| P(BtT-HT-T)-10-30- | 10-30-60 | 10-31-59 | 15209 | 1.23 | −9 |
| 60 | | | | | |

### In Vitro Hydrolytic Degradation

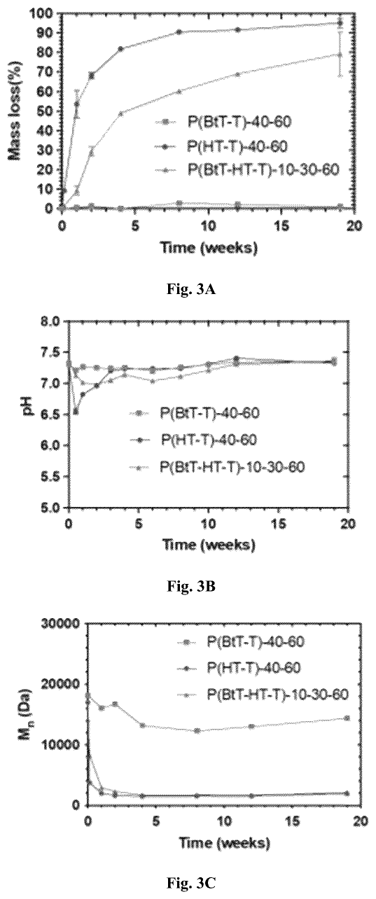

The substitution of the pendant hydroxyl group with a butyrate group produced a notable change in the degradation behavior of the polymer. While un-substituted P(HT-T)-40-60 degraded rapidly, with 53.4±6.6% mass loss by 1 week, 81.8±0.4% mass loss in 4 weeks, and nearly complete mass loss by 19 weeks, the fully butyrate substituted P(BtT-T)-40-60 exhibited negligible mass loss over the 19 weeks (FIG. 3A). The P(BtT-HT-T)-10-30-60 exhibited an intermediate degradation rate, reaching a mass loss of 49±1% by 4 weeks, and 79±8% by 19 weeks. Importantly, the pH of the degradation medium only decreased slightly, remaining above 6.5 for the duration of the degradation study for all polymers (FIG. 3B). The largest decrease in pH was exhibited by the P(HT-T)-40-60, while the P(BtT-HT-T)-10-30-60 generated a lesser decrease in medium pH but a more gradual return to the initial pH. The pH of the P(Bt-T)-40-60 remained unchanged throughout the study. Both the P(BtT-HT-T)-10-30-60 and the P(HT-T)-40-60 exhibited a rapid decrease in number average molecular weight (Mₙ) initially, with decreases to 58 and 22% of the initial Mₙ over the first day and 21 and 12% by the first week, respectively (FIG. 3C). By week 2, the Mₙ of the remaining material reached plateau values of 1560±180 Da for the P(HT-T)-40-60, and 1940±280 Da for the P(BtT-Ht-T)-10-30-60. The Mₙ of the P(BtT-T)-40-60 decreased over the first 4 weeks although it did not exhibit any mass loss, indicating that some bond cleavage did occur but that the products formed were not soluble in the PBS. In support of this explanation, the dispersity of the P(Bt-T)-40-60 increased from an initial value of 1.33 to 1.51 by week 19.

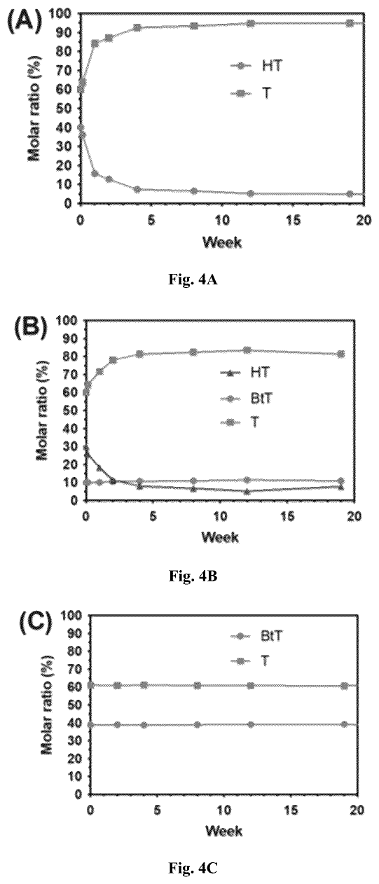

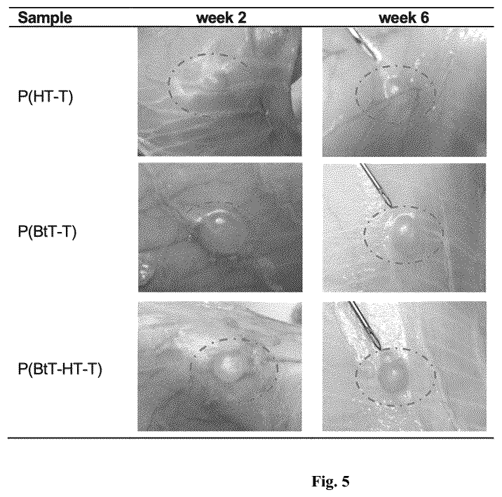

The change in the molar ratio of HT in the polymers reflected their mass loss, with a faster decrease in HT composition accompanying a greater mass loss. For example, P(HT-T)-40-60 decreased in HT molar ratio from 40% initially to 7.5±1% by week 4 (FIG. 4A), at which point the polymer had lost 81.8±0.4% of its initial mass (FIG. 3A), and P(BtT-HT-T)-10-30-60 decreased in HT molar ratio from 30% initially to 8±1% by week 4 (FIG. 4B), over which time it had lost 49±1% of its initial mass. Moreover, throughout the degradation period, the BtT composition of the remaining P(BtT-HT-T)-10-30-60 did not change, remaining at 10±1 mol/mol %. Finally, consistent with its lack of mass loss, the P(BtT-T)-40-60 did not change in monomer composition throughout the study period (FIG. 4C).

It has been shown that P(HT-T) degradation proceeds through a nucleophilic attack of an activated pendant hydroxyl group on a nearby carbonyl group, generating lower molecular weight polymer chains with a glycerol carbonate five-membered ring end group (Chen et al., 2018; Mohajeri et al., 2020), while the carbonate bond between trimethylene carbonate bonds is stable. This intramolecular cyclization reaction is base-catalyzed and occurs within hours. The pendant glycerol carbonate end groups formed hydrolyse slowly, to form carbon dioxide and a terminal HT group. The terminal HT groups undergo intramolecular cyclization producing free glycerol carbonate, which then hydrolyses to glycerol and carbon dioxide. Some of the carbon dioxide reacts with water to form the weak acid carbonic acid. Ultimately, the degradation products are glycerol, carbon dioxide, carbonic acid and low molecular weight P(HT-T) with very low HT content. Mass loss is observed if the low molecular weight P(HT-T) products are soluble in the surrounding medium. From FIG. 3C, the Mₙ below which P(HT-T) with an HT content of 8±1% is soluble in PBS at 37° C. is 1560±180 Da. This value is consistent with the limit found previously of 1350±300 Da for P(HT-T) with an HT content of 10±2% (Mohajeri et al., 2020).

However, the degradation mechanism of P(BtT-HT-T) is different. Partial conversion of the pendant hydroxyl groups to butyrate groups resulted in a slower degradation rate as fewer backbone cleavage reactions initially occurred by intramolecular cyclization caused by HT repeating units. Moreover, as suggested by the data in FIG. 4B, the ester group linking the butyrate to the backbone was stable over the time period examined. Thus the remaining polymer fragments contained pendant butyrate groups, which reduced the water solubility of polymer products of a given molecular weight. As such, the slower mass loss of the P(BtT-HT-T)-10-30-60 was a result of fewer backbone cleavage reactions and a slower dissolution rate of the low molecular polymer products formed. Complete conversion of the pendant hydroxyl groups of the P(BtT-T)-40-60 blocked degradation via intramolecular cyclization, with the resulting aliphatic polycarbonate being resistant to hydrolysis, exemplified by no mass loss, and only a small decrease in Mₙ and increase in dispersity. As one of the degradation products is carbon dioxide, which is partially converted to carbonic acid in the aqueous medium, the faster degradation rate and accompanying carbonic acid formation is the reason for the greater change in degradation medium pH for the P(HT-T) and the more moderate but more persistent reduction in medium pH of the P(BtT-HT-T)-10-30-60 and the lack of a change in medium pH of the P(BtT-T)-40-60.

### In Vivo Degradation and Tissue Response

The properties of the polymers used in the in vivo studies are given in Table 3. Polymers were prepared of varying composition to assess the influence of the butyrate group on the foreign body response. Following implantation, all the animals gained weight and showed no sign of discomfort or adverse responses, such as sores on the skin, throughout the study period. A photograph of the surrounding tissue was taken after exposure of the injection site, and the tissue around the injection site was visually assessed. FIG. 5 shows representative images of the polymer samples within the subcutaneous tissue at weeks 2 and 6. There was no evidence of adverse tissue response around any of the implanted polymers.

TABLE 3
Physico-chemical properties summary of the
polymers prepared for the in vivo studies.

| | Monomer molar ratio | | |
|---|---|---|---|
| Sample | BtT-HT-T | Mₙ (Da) | Ð |
| P(HT-T)-30-70 | 0-30-70 | 3815 | 1.35 |
| P(BtT-HT-T)-15-25-60 | 15-23-62 | 9322 | 1.28 |
| P(BtT-T)-40-60 | 38-0-62 | 10081 | 1.37 |

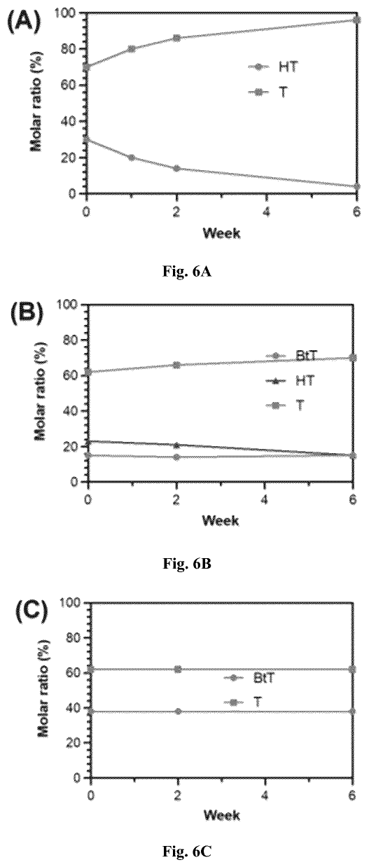

Due to the difficulty of separating the polymer from the surrounding tissue completely, the mass loss could not be evaluated. However, samples were taken and analyzed via 1H NMR spectroscopy for composition. No peaks apart from the expected polymer peaks were observed. The HT molar ratio for both poly(HT-T)-30-70 and P(BtT-HT-T)-15-25-60 decreased similarly as in the in vitro study (FIGS. 6A and 6B) while the molar composition for P(BtT-T)-40-60 did not change appreciably over the 6 weeks examined (FIG. 6C). Moreover, the BtT content of the retrieved P(BtT-HT-T)-15-25-60 samples remained constant (FIG. 6B), as was found in the in vitro degradation study.

### Macrophage Polarization

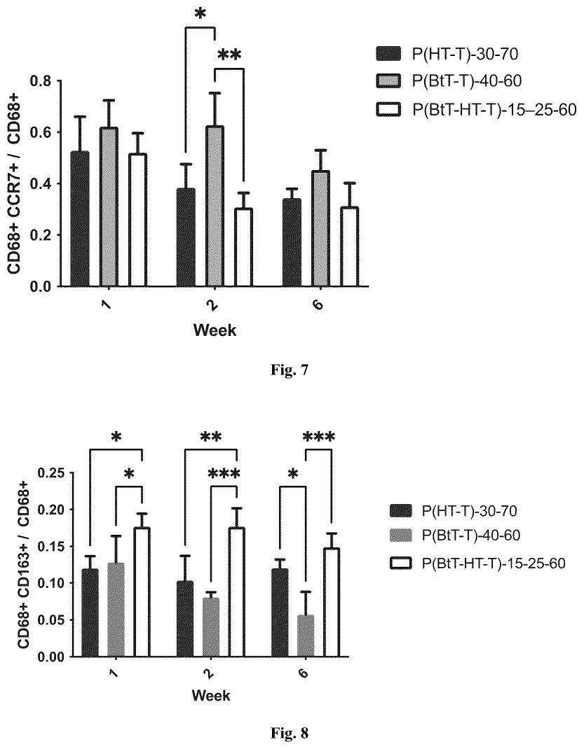

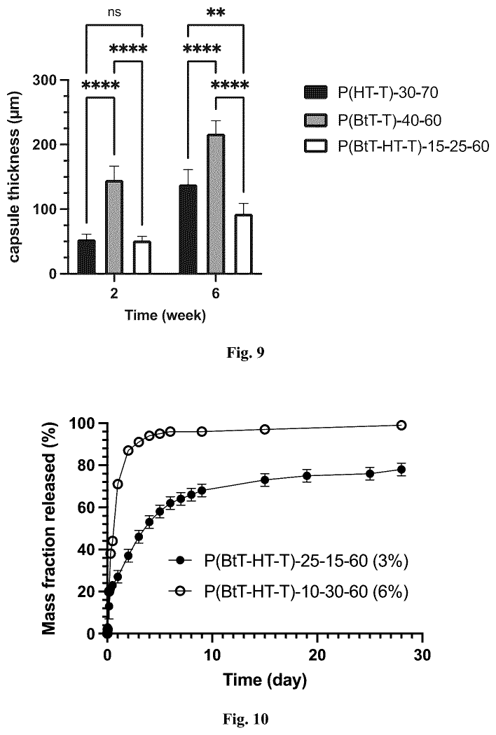

Tissue around the implanted polymers was excised, sectioned and stained with CD68, which is a pan macrophage marker, CCR7 which is a marker for M1 macrophages, and CD163, which is a marker for M2 macrophages (Witherel et al., 2019). Cells within the inflammatory zone around the implants that were CD68+CCR7+ were considered M1 macrophages while cells that were CD68+CD163+ were considered M2 macrophages. The number of M1 and M2 macrophages were counted and are presented as the ratio of M1 macrophages to total macrophages (CD68+CCR7+/CD68+) (FIG. 7) and the ratio of M2 macrophages to total macrophages (CD68+CD163+/CD68+) (FIG. 8) with time. Other than at week 2, where the fraction of M1 macrophages present in the inflammatory zone was higher for the P(BtT-T)-40-60 implants, there was no significant difference in the fraction of M1 macrophages present for any of the polymer implants. However, the fraction of M2 macrophages present in the inflammatory zone was significantly greater for the P(BtT-HT-T)-15-25-60 implants than the other two copolymers at weeks 1 and 2, and than the P(BtT-T)-40-60 implants at week 6.

### Capsule Thickness

The rate of formation of a fibrous capsule around the implants was determined from assessments of Masson's trichrome stained tissue sections in which cell nuclei stain black, collagen stains blue, and muscle, erythrocytes, and cell cytoplasm stains red. A zone of inflammation is apparent by week 1 with high cell density but no organized collagen fibre formation. A thin fibrous capsule delineated by organized collagen fibres is present around all the polymers by week 2 and persists to week 6. The thickness of the capsule formed was significantly greater at week 6 around the P(HT-T)-30-70 and P(BtT-T)-40-60 than around the P(BtT-HT-T)-15-25-60. The thinner capsule reflects the reduced fibrotic response expected of having a higher M2 population density around the implants. Moreover, the non-degradable P(BtT-T)-40-60 had the thickest capsule at all time points.

Collectively, the polymer degradation and histology data indicate that butyrate does induce macrophage polarization towards an M2 phenotype in tissue but that the butyrate must be liberated from the polymer backbone to do so. Contrary to expectations based on previous work, the butyrate is not liberated through hydrolysis of the ester group linking it to the polymer backbone, but from an intramolecular cyclization reaction which cleaves the polymer backbone forming soluble oligomers bearing the pendant butyrate group.

### In Vitro Peptide Release

As an example of the utility of butyrated aliphatic polycarbonates according to embodiments described herein, the sustained release of a representative drug, octreotide acetate, was demonstrated. For this purpose, polymers were prepared having the properties given in Table 4.

TABLE 4
Composition and molecular weight and dispersity of
polymers prepared for in vitro peptide release.

| | Monomer molar ratio | | |
|---|---|---|---|
| Sample | BtT-HT-T | Mₙ (Da) | Ð |
| P(BtT-HT-T)-25-15-60 | 24-15-61 | 3440 | 1.15 |
| P(BtT-HT-T)-10-30-60 | 11-30-59 | 4000 | 1.20 |

Sustained octreotide release from 1 to 4 weeks was achieved, at rates controllable by manipulating the composition of the P(BtT-HT-T) used. Results are shown in FIG. 10. Release was slower from polymers having a greater % of BtT repeating units. For example, octreotide release from P(BtT-HT-T)-10-30-60 was rapid, with 71±3% of the initially loaded mass released by 24 h and 96±1% released by 6 days. By contrast, octreotide release from P(BtT-HT-T)-25-15-60 was slower, with 27±3% released by 24 h, 62±3% released by 6 days, and 78±3% released by 28 days. The difference in release is a result of a combination of a reduction in water penetration into the polymer bulk with higher BtT composition, due to its lower water solubility, and a reduction in the degradation rate of the polymer caused by fewer HT repeating units.

### INCORPORATION BY REFERENCE

The contents of all cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

### REFERENCES

Chen, F., et al., Degradation of poly(5-hydroxy-trimethylene carbonate) in aqueous environments. Polymer Degradation And Stability, 158: 83-91, 2018.
Cox, M. A., et al., Short-chain fatty acids act as antiinflammatory mediators by regulating prostaglandin E2 and cytokines. World Journal of Gastroenterology: WJG 15: 5549-5557, 2009.
Klopfleisch, R. and F. Jung, The pathology of the foreign body reaction against biomaterials. Journal Of Biomedical Materials Research Part A, 105(3): 927-940, 2017.
Mohajeri, S., et al., Liquid degradable poly(trimethylene-carbonate-co-5-hydroxy-trimethylene carbonate): An injectable drug delivery vehicle for acid-sensitive drugs. Molecular Pharmaceutics, 17(4): 1-14, 2020.
Tedelind, S., F., et al., Antiinflammatory properties of the short-chain fatty acids acetate and propionate: A study with relevance to inflammatory bowel disease. World Journal of Gastroenterology 13: 2826, 2007.
Vinolo, M. A. R., et al., Suppressive effect of short-chain fatty acids on production of proinflammatory mediators by neutrophils. The Journal of Nutritional Biochemistry 22: 849-855, 2010.
Vinolo, M. A. R., et al., Effects of short chain fatty acids on effector mechanisms of neutrophils. Cell Biochemistry and Function 27: 48-55, 2009.
Witherel, C. E., et al., Macrophage and Fibroblast Interactions in Biomaterial-Mediated Fibrosis. Advanced Healthcare Materials, 8(4), 2019.

1. A composition, comprising:
a polymer having a polymer backbone bearing pendant groups wherein at least a portion of the pendant groups comprise short chain fatty acid groups;
wherein the short chain fatty acid groups are conjugated to the pendant groups of the polymer backbone by degradable linkages.

2. The composition of claim 1, wherein the degradable linkages are degradable by one or more of hydrolysis, oxidation, and enzymatic action.

3. The composition of claim 1, wherein the degradable linkages comprise one or more of ester, anhydride, carbonate, amide, thioester, urethane, acetal, disulfide, and orthoester.

4. The composition of claim 1, wherein the degradable linkages are ester linkages.

5. The composition of claim 1, wherein the portion of the pendant groups that are conjugated with short chain fatty acid groups is from 1% to 100%.

6. The composition of claim 1, wherein the short chain fatty acid groups comprise one or more of acetate, propionate, and butyrate.

7. The composition of claim 1, wherein the polymer is selected from a vinyl polymer, a polyester, and an aliphatic polycarbonate.

8. The composition of claim 1, wherein the polymer is an aliphatic polycarbonate.

9. The composition of claim 1, wherein the polymer is selected from poly(vinyl alcohol), poly(5-hydroxy caprolactone), poly(5-hydroxy caprolactone-co-lactide) and combinations with glycolide and/or trimethylene carbonate, poly(5,6-hydroxy tetramethylene carbonate), poly(5,5-dihydroxy trimethylene carbonate), and poly(1,2-glycerol carbonate).

10. The composition of claim 1, wherein the polymer is degradable.

11. The composition of claim 10, wherein a degradation rate of the polymer is controlled according to a molar ratio of repeating unit in the polymer.

12. The composition of claim 10, wherein the polymer is poly(2-hydroxy trimethylene carbonate-trimethylene carbonate) (poly(HT-T)).

13. The composition of claim 1, wherein the short chain fatty acid groups comprise butyrate.

14. The composition of claim 13, comprising:
2-butyrate trimethylene carbonate (BtT);
2-hydroxy trimethylene carbonate (HT); and
trimethylene carbonate (T).

15. The composition of claim 14, wherein the BtT, HT, and T are present in a ratio within the range of about 25-15-60 to about 10-30-60.

16. The composition of claim 1, wherein the composition comprises a biomedical device that is implantable and degradable in a subject.

17. The composition of claim 1, wherein degradation of the degradable linkage liberates groups comprising at least a portion of the short chain fatty acid groups from the polymer backbone.

18. The composition of claim 1, wherein degradation of the degradable linkage comprises an intramolecular cyclization reaction that cleaves the polymer backbone forming soluble oligomers bearing the short chain fatty acid groups.

19. The composition of claim 16, wherein degradation of the degradable linkage liberates groups comprising at least a portion of the short chain fatty acid groups from the polymer backbone;
wherein the groups comprising at least a portion of the short chain fatty acid groups liberated from the polymer backbone induce an immunomodulatory response in the subject.

20. The composition of claim 19, wherein the immunomodulatory response in the subject comprises macrophage polarization towards an M2 phenotype in tissue.

21. The composition of claim 16, wherein the biomedical device comprises a drug delivery vehicle and the composition further comprises at least one drug.

22. The composition of claim 21, wherein the at least one drug comprises at least one of a therapeutic compound, a pharmaceutical agent, a biopharmaceutical agent, a bioactive agent, a medicament, an antineoplastic agent, a hormone, a peptide, a protein, a nucleic acid, a vector, a virus, an antigen, and an antibody.

23. An implantable biomedical device comprising the composition of claim 1.

24. A method for delivering one or more short chain fatty acid groups to a subject, comprising:
implanting in an anatomical site, organ, or tissue of the subject the composition of claim 1;
wherein degradation of the degradable linkages liberates groups comprising at least a portion of the short chain fatty acid groups from the polymer backbone and delivers the at least a portion of the short chain fatty acid groups to the anatomical site, organ, or tissue of the subject.

25. A method for resolving inflammation, promoting tissue healing, and/or promoting tolerating self-antigens and neoantigens in a subject, comprising:
implanting in an anatomical site, organ, or tissue of the subject the composition of claim 1;
wherein degradation of the degradable linkages liberates groups comprising at least a portion of the short chain fatty acid groups from the polymer backbone and delivers the at least a portion of the short chain fatty acid groups to the anatomical site, organ, or tissue of the subject;
wherein the liberated short chain fatty acid groups resolve inflammation, promote tissue healing, and/or promote tolerating self-antigens and neoantigens in the anatomical site, organ, or tissue of the subject.
